(19) United States
(12) Patent Application Publication
(10) Pub. No.: US 2024/0066006 A1
(43) Pub. Date: Feb. 29, 2024

(54) Antiviral Compounds and Applications Thereof

(71) Applicant: The Regents of the University of California, Oakland, CA (US)

(72) Inventors: Aimee L. Edinger, Irvine, CA (US); Brendan T. Finicle, Anaheim, CA (US)

(73) Assignee: The Regents of the University of California, Oakland, CA (US)

(21) Appl. No.: 18/258,122

(22) PCT Filed: Dec. 17, 2021
(86) PCT No.: PCT/US21/73013

§ 371 Date: Jun. 16, 2023

Related U.S. Application Data
(60) Provisional application No. 63/127,009, filed on Dec. 17, 2020.

(51) Int. Cl. A61K 31/40; A61K 31/4196; A61K 31/519; A61K 31/5377; A61P 31/14

(52) U.S. Cl. CPC A61K 31/40; A61K 31/4196; A61K 31/519; A61K 31/5377; A61P 31/14

(57) ABSTRACT

Antiviral compounds can be utilized to mitigate viral activity of enveloped viruses in an infected host. In some instances, a virally infected biological cell is contacted with an antiviral to mitigate viral activity in the biological cell. In some instances, a virally infected animal is administered an antiviral to mitigate viral activity in the animal. In some instances, an animal is prophylactically administered an antiviral to mitigate viral activity in the animal. Therapeutics and treatments involving antiviral compounds are also described.

### CROSS REFERENCE TO RELATED APPLICATIONS

This application claims priority to U.S. Provisional Application Ser. No. 63/127,009 entitled “Antiviral Compounds and Applications Thereof,” by Aimee L. Edinger et al., filed Dec. 17, 2020, which is herein incorporated by reference in its entirety.

### TECHNICAL FIELD

The disclosure is generally directed to antiviral compounds, including compounds that agonize PP2A activity, and applications thereof, including antiviral treatments.

### BACKGROUND

A virus is a submicroscopic infectious agent that replicates and propagates within a host biological cell. That is, unlike bacteria and other cellular based microorganisms, viruses require a host cell to make more viruses. Several different classes of viruses exist, each having definitive characteristics. For example, several viruses utilize a protein coat or capsid to surround its genetic material; these viruses are referred to by their capsid shape such as “icosahedral viruses” or “helical viruses.” Adenovirus, picornavirus, rotavirus and rhinovirus are common icosahedral viruses. Several other viruses utilize an envelope of lipids to surround its genetic material; these viruses are referred to as “enveloped viruses.” Enveloped viruses require the host's endocytic and exocytic pathways to form infectious virions. Enveloped viruses include coronavirus (e.g., severe acute respiratory syndrome corona virus 2 (SARS-CoV-2)), herpesvirus (e.g., chicken pox), poxvirus (e.g., small pox), retrovirus (e.g, human immunodeficiency virus (HIV)), flavivirus (e.g., dengue virus, zika virus), hepadnavirus (e.g., hepatitis B), pneumovirus (e.g., respiratory syncytial virus (RSV)), influenza virus, ebolavirus, rabies virus, mumps virus, and papillomavirus.

Antivirals are a class of medication for treating viral infections. Most antivirals target a specific viral protein. For example, antiretroviral therapy (ART) for HIV targets three HIV-specific proteins: a protease, reverse transcriptase, and integrase. Therapies for coronavirus disease 2019 (COVID-19), which is caused by SARS-CoV-2 infection, include remdesivir, which targets the RNA-dependent RNA polymerase that is essential for viral genome replication. Many groups are also developing monoclonal antibodies that target the SARS-CoV-2 spike protein that is essential for cell entry. Because these therapies target specific viral components, viruses can develop mutations that confer resistance to these therapies.

Additionally, these therapies are highly specific for each virus. Thus, researchers and clinicians must re-invent therapies for each emerging virus. Because of these limitations, there is an unmet critical need for novel antivirals that have broad-spectrum activity against many enveloped viruses.

### SUMMARY

The present disclosure provides antiviral compounds and methods of use to mitigate viral activity. In certain embodiments, the disclosure provides methods for agonizing protein phosphatase 2A (PP2A) activity, resulting in reducing viral replication by reducing endocytic trafficking to the plasma membrane, endosomal acidification, and fusion with lysosomes. In certain embodiments, antiviral compounds are utilized in treatments against viral infection.

In an embodiment, viral activity is mitigated in a biological cell by contacting the biological cell with an antiviral compound. The antiviral compound is an agonist of protein phosphatase 2 (PP2A) or an antagonist of ADP Ribosylation Factor 6 (ARF6). The biological cell is infected with an enveloped virus or at risk of being infected with an enveloped virus.

In an embodiment, subject is treated for an active viral infection or for preventing a viral infection by administering to the subject an antiviral compound. The antiviral is an agonist of protein phosphatase 2 (PP2A) or an antagonist of ADP Ribosylation Factor 6 (ARF6). The subject is infected with an enveloped virus or at risk of infection with an enveloped virus.

In an embodiment, an antiviral compound is used in the manufacture of a medicament for the therapeutic treatment of an infection with an enveloped virus. The antiviral compound is an agonist of protein phosphatase 2 (PP2A) or an antagonist of ADP Ribosylation Factor 6 (ARF6).

### BRIEF DESCRIPTION OF THE DRAWINGS

The description and claims will be more fully understood with reference to the following figures and data graphs, which are presented as exemplary embodiments and should not be construed as a complete recitation of the scope of the disclosure.

FIGS. 1A, 1B, and 1C provide an experimental timeline schematic, data results, and microscopy images of the effect of pretreatment with antiviral compound 893 on viral activity using DBT cells infected with MHV-1 and MHV-A59, generated in accordance with various embodiments.

FIGS. 2A, 2B, and 2C provide an experimental timeline schematic, data results, and microscopy images of the effect of treatment with antiviral compound 893 on virus replication using DBT cells infected with MHV-1 and MHV-A59, generated in accordance with various embodiments.

FIGS. 3A, 3B, and 3C provide data results of and microscopy images depicting the effect of antiviral compound treatment on syncytium formation using murine astrocytoma cells (DBT) infected with the murine coronavirus MHV, generated in accordance with various embodiments.

FIGS. 4A to 4D provide an experimental schematic and data results of the effect of treatment of antiviral compound 893 on syncytium formation using HeLa, HEK-293, and VeroE6 cells as assessed by GFP fluorescence, generated in accordance with various embodiments.

FIG. 4E provides a fluorescent microscopy image depicting the localization of the ACE2 receptor in VeroE6 cells treated with antiviral compound 893, generated in accordance with various embodiments.

FIG. 5 provides a schematic of enveloped virus lifecycle and the relationship to host factors PIKfyve, ARF6, and PP2A, utilized in accordance with various embodiments.

FIGS. 6A, 6B, and 6C provide an experimental timeline schematic, data results, and microscopy images of the effect of pretreatment with antiviral compound 893 or a PIKfyve antagonist on viral activity using DBT cells infected with MHV-1, generated in accordance with various embodiments.

FIGS. 7A, 7B, and 7C provide an experimental timeline schematic, data results, and microscopy images of the effect of pretreatment with antiviral compound 893 or an ARF6 antagonist on viral activity using DBT cells infected with MHV-1, generated in accordance with various embodiments.

FIGS. 8A and 8B provide data results of the effect of pretreatment with a PIKfyve antagonist and an ARF6 antagonist on viral activity using DBT cells infected with MHV-1 or MHV-A59, generated in accordance with various embodiments.

FIGS. 9A, 9B, and 9C provide an experimental timeline schematic, data results, and microscopy images of the effect of pretreatment with PP2A agonists PPZ or compound 893 on viral activity using DBT cells infected with MHV-1, generated in accordance with various embodiments.

FIGS. 10A and 10B provide an experimental timeline schematic and data results of the effect of treatment with compound 893 in mice infected with MHV-1, generated in accordance with various embodiments.

### DETAILED DESCRIPTION

Turning now to the drawings and data, antagonistic compounds of viral activity of enveloped viruses, and methods of their use are described in accordance with the various embodiments. In some embodiments, an antagonist of viral activity is utilized to mitigate the activity of an enveloped virus in infected cells. In some embodiments, a virally infected biological cell is contacted with an antiviral compound to mitigate the viral activity in that biological cell. In some embodiments, an animal is administered an antiviral compound to mitigate viral activity in the animal. In some embodiments, an antiviral compound is used in a course of treatment. In some embodiments, an antiviral compound is utilized in the manufacture of a medicament for the therapeutic treatment of a viral infection.

Viruses require a biological cell of a host to replicate. To enter the host cell and gain access to the host's cellular machinery to build new virions, viruses hijack cellular highways that are used to transport nutrients and other material into cells (i.e., endocytic pathways). Once inside the host cell, cellular highways are used as conveyor belts to facilitate the assembly and release of new virions (i.e., via exocytic pathways). Several compounds have been identified that block these trafficking pathways, shutting down both viral entry and exit. Many enveloped viruses (e.g., coronaviruses, influenza, hepatitis, HPV, Ebola, Zika, etc), utilize these cellular highways for entry and exit, and drugs with this action could be effective in a wide range of viral diseases. Accordingly, it is proposed that targeting host cell intracellular trafficking will block viral entry and/or replication of enveloped viruses. History supports that novel coronavirus or novel influenza epidemics will continue to occur. A novel therapy that inhibits the coronavirus or influenza replication cycle rather than specific viral components would allow for treatments of any novel enveloped viruses immediately upon emergence.

In accordance with various embodiments, small molecules that agonize protein phosphatase 2A (PP2A) will be effective antivirals able to slow the replication of many enveloped viruses. PP2A activation can mitigate replication of many enveloped viruses, including (but not limited to) coronavirus (e.g., severe acute respiratory syndrome corona virus 2 (SARS-CoV-2)), herpesvirus (e.g., chicken pox), poxvirus (e.g., small pox), retrovirus (e.g., human immunodeficiency virus (HIV)), flavivirus (e.g., dengue virus, zika virus), hepadnavirus (e.g., hepatitis B), pneumovirus (e.g., respiratory syncytial virus (RSV)), influenza virus, ebolavirus, rabies virus, mumps virus, and papillomavirus. In various embodiments, small molecules that can agonize PP2A include (but are not limited to) sphingolipids, sphingolipid-like compounds, sphingolipid-like compound 893, sphingolipid-like compound 1090, sphingolipid-like compound 325, ceramide, perphenazine, perphenazine derivatives (e.g., small molecule activator of PP2A (SMAP or DT-061), iHAP (2-chloro-10-(4-methoxybenzoyl)-10H-phenothiazine)), SET inhibitors, CIP2a inhibitors, Withaferin A, OSU-2S, and derivatives thereof.

PP2A activation can mitigate the replication of enveloped viruses. For example, the coronavirus infection cycle consists of 3 steps: 1) entry following receptor recognition and membrane fusion mediated by viral spike protein cleavage at the plasma membrane or in acidified endosomes, 2) viral genome replication and viral protein production in the cytosol, and 3) release of new virus assembled in the ER-Golgi via exocytosis or lysosome exocytosis. Coronaviruses can also spread to neighboring host cells by causing cell-cell fusion. This phenomenon is called syncytium formation and occurs by cell surface expression of the viral spike protein. ADP Ribosylation Factor 6 (ARF6) is a small GTPase that is essential for endocytic recycling and thus also for cell surface expression of many proteins. Additionally, ARF6 is also necessary for trafficking from the Golgi network to the plasma membrane. Therefore, antagonizing ARF6 may lower cell surface expression of viral receptor proteins (e.g. ACE2), cell surface proteases that promote entry (e.g. TMPRSS2), and the viral spike protein and impede the trafficking of assembled viral particles from the ER-Golgi to the cell surface and/or exocytosis via lysosomes. ARF6 inhibition may also disrupt localization of intracellular proteases like furin that contribute to the activation of viral spike proteins, thereby limiting viral infectivity. Endosomal acidification can promote spike-mediated membrane fusion and requires maturation of early endosomes and fusion with lysosomes mediated by the lipid kinase PIKfyve. Activation of PP2A simultaneously inactivates ARF6 and causes PIKfyve disfunction. Therefore, small molecules that activate PP2A have the potential to mitigate enveloped virus replication cycle at multiple steps.

### Definitions

Unless otherwise indicated, the following terms have the following meanings:

“Antiviral activity” refers to a mitigation or inhibition of any viral activity, including (but not limited to) viral replication, hijacking of host cellular machinery, inhibition of a host's antiviral response, syncytia, and virulence and does not necessarily indicate a total elimination of viral activity.

“Syncytium” refers to the fusion of two biological cells. Syncytia can arise when cells are infected with virus, especially enveloped viruses, as viral spike proteins are transported to the host cell surface where they can interact with their cell surface receptors causing membrane fusion.

“Viral infection” refers to a virus or a virus derivative (e.g., viral vector) inside of a biological cell or the body of animal and includes any route of entry into the cell or the body.

“Pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by a subject. In certain embodiments, a pharmaceutically acceptable carrier or diluent is sterile water; sterile saline; or sterile buffer solution.

“Pharmaceutically acceptable salts” means physiologically and pharmaceutically acceptable salts of compounds, such as antiviral compounds, i.e., salts that retain the desired biological activity of the parent compound and do not impart undesired toxicological effects thereto.

“Pharmaceutical composition” means a mixture of substances suitable for administering to a subject. For example, a pharmaceutical composition may comprise an antiviral compound and a sterile aqueous solution.

“Prodrug” means a therapeutic agent in a form outside the body that is converted to a different form within the body or cells thereof. Typically conversion of a prodrug within the body is facilitated by the action of an enzymes (e.g., endogenous or viral enzyme) or chemicals present in cells or tissues and/or by physiologic conditions.

“Acyl” means a —R—C═O group.

“Alcohol” means a compound with an —OH group bonded to a saturated, alkane-like compound, (ROH).

“Alkyl” refers to the partial structure that remains when a hydrogen atom is removed from an alkane.

“Alkane” means a compound of carbon and hydrogen that contains only single bonds.

“Alkene” refers to a hydrocarbon that contains a carbon-carbon double bond, R₂C═CR₂.

“Alkyne” refers to a hydrocarbon structure that contains a carbon-carbon triple bond.

“Alkoxy” refers to a portion of a molecular structure featuring an alkyl group bonded to an oxygen atom.

“Aryl” refers to any functional group or substituent derived from an aromatic ring.

“Amine” molecules are compounds containing one or more organic substituents bonded to a nitrogen atom, RNH₂, R₂NH, or R₃N.

“Amino acid” refers to a difunctional compound with an amino group on the carbon atom next to the carboxyl group, RCH(NH₂)CO₂H.

“Azide” refers to N₃.

“Cyanide” refers to CN.

“Ester” is a compound containing the —CO₂R functional group.

“Ether” refers to a compound that has two organic substituents bonded to the same oxygen atom, i.e., R—O—R′.

“Halogen” or “halo” means fluoro (F), chloro (Cl), bromo (Br), or iodo (I).

“Hydrocarbon” means an organic chemical compound that consists entirely of the elements carbon (C) and hydrogen (H).

“Phosphate”, “phosphonate”, or “PO” means a compound containing the elements phosphorous (P) and oxygen (O).

“R” in the molecular formula above and throughout are meant to indicate any suitable organic molecule.

### Certain Antiviral Compounds

In certain embodiments, protein phosphatase 2 (PP2A) agonists are antiviral compounds (i.e., antagonists of viral activity). In certain embodiments, PP2A agonists are contacted with a biological cell infected with virus or a biological cell prior to infection with virus, which can mitigate and/or inhibit viral activity within that cell. As discussed herein, PP2A agonists inhibit endocytic and exocytic trafficking, endosomal acidification, and fusion of lysosomes of a biological cell, which are activities that boost virus replication and propagation. PP2A agonists include (but are not limited to) sphingolipids, sphingolipid-like compounds, sphingolipid-like compound 893, sphingolipid-like compound 1090, sphingolipid-like compound 325, ceramide, perphenazine, perphenazine derivatives (e.g., small molecule activator of PP2A (SMAP or DT-061), iHAP (2-chloro-10-(4-methoxybenzoyl)-10H-phenothiazine)), SET inhibitors, CIP2a inhibitors, Withaferin A, OSU-2S, and derivatives thereof. Numerous PP2A agonists are described in the literature and can be utilized in certain embodiments as described herein (see A. R. Clark and M Ohlmeyer, Pharmacol Ther. 2019; 201:181-201; M. R. Carratu, et al., Curr Med Chem. 2016; 23(38):4286-4296; M. Remmerie and V. Janssens, Front Oncol. 2019; 9:462; D. B. Kastrinsky, et al., Bioorg Med Chem. 2015; 23(19):6528-6534; and K. McClinch, et al., Cancer Res. 2018; 78(8):2065-2080; the disclosures of which are incorporated herein by reference).

Sphingolipids and PP2A agonists inactivate ADP Ribosylation Factor 6 (ARF6). In certain embodiments, ARF6 antagonists are antagonists of viral activity. In certain embodiments, ARF6 antagonists are applied to a biological cell infected with virus to mitigate and/or inhibit viral activity within that cell. As discussed herein, ARF6 is involved with endocytic recycling and thus antagonists of ARF6 can mitigate virus release from cells and can reduce ectopic expression of viral proteins involved in syncytium formation. ARF6 antagonists include (but are not limited to) sphingolipids, sphingolipid-like compounds, sphingolipid-like compound 893, sphingolipid-like compound 1090, sphingolipid-like compound 325, sphingolipid-like compound LS-200, ceramide, NAV2729, SecinH3, perphenazine, and derivatives thereof. Numerous ARF6 antagonists are described in the literature and can be utilized in certain embodiments as described herein (see B. T. Finicle, et al., J Cell Sci. 2018; 131(12):jcs213314; J. H. Yoo, et al., Cancer Cell. 2016; 29(6):889-904; and M. Hafner, et al., Nature. 2006; 444(7121):941-944; the disclosures of which are incorporated herein by reference).

In certain embodiments, an antiviral is applied prior to, concurrently with, or after viral infection in a biological cell. Accordingly, in certain embodiments, prior to infection with a virus, a biological cell is contacted with an antiviral compound. In certain embodiments, after infection with a virus, a biological cell is contacted with an antiviral compound. In certain embodiments, prior to infection of a virus, an animal is administered an antiviral compound (e.g., prophylactic administration). In certain embodiments, after infection with a virus, an animal is administered an antiviral compound to treat the viral infection.

In certain embodiments, an antiviral compound is utilized at concentration between 1 nM to 100 μM. In various embodiments, an antiviral compound is utilized at a concentration less than 1 nM, approximately 1 nM, approximately 10 nM, approximately 100 nM, approximately 1 μM, approximately 10 μM, approximately 100 μM, or greater than 100 μM. When referring to concentrations, “approximately” is to be interpreted as within an order of magnitude (e.g., “approximately 1 nM” is 1 nM to less than 10 nM).

### I. Sphingolipid-Like Compounds

### A. Sphingolipid-Like Compounds Based on O-benzyl Azacycles

In certain embodiments, an antiviral compound is based on O-benzyl azacycles. In certain embodiments, an antiviral compound is of formula:

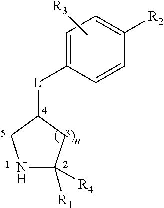

R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, wherein an akyl, alkyne, or alkene is an aliphatic chain up to ten carbons;

R₂ is an aliphatic chain (C₆-C₁₀);

R₃ is a mono-, di-, tri- or quad-aromatic substituent comprising H, halogen, alkyl, alkoxy, azide (N₃), ether, NO₂, or cyanide (CN);

One of R₁ and R₄ is an alcohol (CH₂OH) or H;

L is O—CH₂; and

n is an independently selected integer selected from 1, 2, or 3.

In certain embodiments of O-benzyl azacycles, the O-benzyl group can be moved to position 4 (shown above) or 3 as shown below:

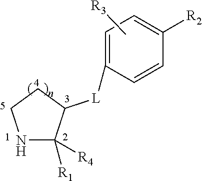

In certain embodiments, alkyl, CH₂OH, or (CH₂)ₙOH groups can be added to position 5.

In certain embodiments, one of R₁ or R₄ is an alkyl having 1 to 6 carbons.

It will be understood that compounds described herein may exist as stereoisomers, enantiomers, diastereomers, cis, trans, syn, anti, solvates (including hydrates), tautomers, and mixtures thereof.

### B. Sphingolipid-Like Compounds Based on 3- and 4-C-aryl Azacycles

In certain embodiments, an antiviral compound is based on diastereomeric 3- and 4-C-aryl azacycles. In certain embodiments, an antiviral compound is of formula:

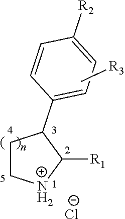

R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, wherein the akyl, the alkyne, or the alkene is an aliphatic chain up to ten carbons;

R₂ is an aliphatic chain (C₆-C₁₄);

R₃ is a mono-, di-, tri- or tetra-aromatic substituent comprising hydrogen, halogen, alkyl, alkoxy, azide (N₃), ether, NO₂, or cyanide (CN); and

n is an independently selected integer selected from 1, 2, or 3.

In certain embodiments of diastereomeric 3- and 4-C-aryl 2-hydroxymethyl azacycles, the C-aryl group can be extended from position 3 (shown above) or position 4 as shown below:

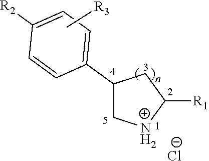

In certain embodiments, alkyl, CH₂OH, or (CH₂)ₙOH groups can be added to position 5.

In certain embodiments, R₂ is an unsaturated hydrocarbon chain.

In certain embodiments, the R₁ is an alkyl having 1 to 6 carbons.

It will be understood that compounds described herein may exist as stereoisomers, enantiomers, diastereomers, cis, trans, syn, anti, solvates (including hydrates), tautomers, and mixtures thereof.

In certain embodiments, an antiviral compound is compound 893, having the formula:

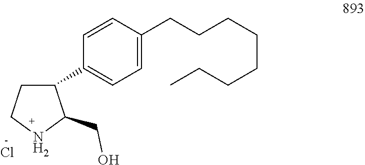 [SMILES: Cl ; CCCCCCCCc1ccc([C@H]2CCN[C@@H]2CO)cc1]

In certain embodiments, an antiviral compound is compound 1090, having the formula:

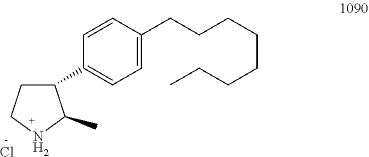 [SMILES: CCCCCCCCc1ccc([C@H]2CCN[C@@H]2C)cc1 ; Cl]

### C. Sphingolipid-Like Compounds Based on Azacycles with Heteroaromatic Appendage

In certain embodiments, an antiviral compound is based on azacycles with an attached heteroaromatic appendage. In certain embodiments, an antiviral compound is of formula:

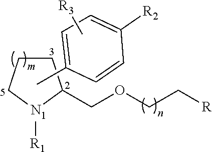

or a pharmaceutically acceptable salt thereof;

R is an optionally substituted heteroaromatic moiety such as an optionally substituted pyridazine, optionally substituted pyridine, optionally substituted pyrimidine, phenoxazine, or optionally substituted phenothiazine.

R₁ is H, alkyl such as C₁₋₆ alkyl or C₁₋₄ alkyl including methyl, ethyl, propyl, isopropyl, n-butyl, s-butyl, isobutyl, t-butyl, etc, Ac, Boc, guanidine moiety.

R₂ is an aliphatic chain comprising 6 to 14 carbons.

R₃ is a 1, 2, 3, or 4 substituents, wherein each substituent, independently, is H, halogen, alkyl, alkoxy, N₃, NO₂, and CN.

n is independently 1, 2, 3, or 4.

m is independently 1 or 2.

The phenyl moiety can be attached at any available position of the azacycle core.

In some embodiments, R₂ is an unsaturated hydrocarbon chain.

In some embodiments, R₂ is C₆₋₁₄ alkyl, C₆₋₁₀ alkyl, C₇₋₉ alkyl, C₆H₁₃, C₇H₁₅, C₈H₁₇, C₉H₁₉, C₁₀H₂₁, C₁₁H₂₃, C₁₂H₂₅, C₁₃H₂₇, or C₁₄H₂₉.

In some embodiments R₃ is H.

In some embodiments, n is 1. In some embodiments, n is 2. In some embodiments, n is 3. In some embodiments, n is 4.

In some embodiments, m is 1. In some embodiments, m is 2.

In some embodiments, the R₂ and R₃ substituents can have different combinations around the phenyl ring with regard to their position.

In some embodiments, the R₁ is an alkyl having 1 to 6 carbons.

It will be understood that compounds described herein may exist as stereoisomers, enantiomers, diastereomers, cis, trans, syn, anti, solvates (including hydrates), tautomers, and mixtures thereof.

In some embodiments, R is a 1,2-pyridazine having the formula:

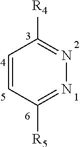

R₄ and R₅ are functional groups independently selected from: alkyl including methyl, optionally substituted aryl (i.e., unsubstituted aryl or substituted aryl) including optionally substituted phenyl, and optionally substituted heteroaryl including optionally substituted pyridine and optionally substituted pyrimidine.

The pyridazine moiety is connected to the azacycle at the position 4 or 5 of the pyridazine.

In some embodiments, any substituents of R₄ and R₅, if present, are independently a halogen (including F), an alkyl, a terminal alkyne, or an azide.

In some embodiments, R₄ is C₁₋₆ alkyl, such as CH₃, C₂ alkyl, C₃ alkyl, C₄ alkyl, C₅ alkyl, or C₆ alkyl; unsubstituted aryl or substituted aryl, including unsubstituted phenyl, or phenyl having 1, 2, 3, 4, or 5 substituents; unsubstituted heteroaryl or substituted heteroaryl, including unsubstituted pyridine or pyridine having 1, 2, 3, or 4 substituents, or unsubstituted pyrimidine or pyrimidine having 1, 2, or 3 substituents. Any substituent may be used in the substituted aryl (e.g., substituted phenyl) or substituted heteroaryl (e.g., substituted pyridine or substituted pyrimidine). For example, the substituents of the substituted aryl or substituted heteroaryl may independently be, halo (such as F, Cl, Br, I), C₁₋₆ alkyl (such as CH₃, C₂ alkyl, C₃ alkyl, C₄ alkyl, C₅ alkyl, C₆ alkyl), or X—R^{a}, wherein X is O, —C(═O)—, —NHC(═O)—, or —C(═O)NH—, and R^{a} is C₁₋₆ alkyl (such as CH₃, C₂ alkyl, C₃ alkyl, C₄ alkyl, C₅ alkyl, C₆ alkyl), C₂₋₆ alkenyl (such as —CH═CH₂, —CH₂CH═CH₂, —CH₂CH₂CH═CH₂, —CH₂CH₂CH₂CH═CH₂, —CH₂CH₂CH₂CH₂CH═CH₂, etc.), or C₂₋₆ alkynyl (such as —CH≡CH₂, —CH₂CH≡CH₂, —CH₂CH₂CH≡CH₂, —CH₂CH₂CH₂CH≡CH₂, —CH₂CH₂CH₂CH₂CH≡CH₂, etc.); or azide.

In some embodiments, R₅ is C₁₋₆ alkyl, such as CH₃, C₂ alkyl, C₃ alkyl, C₄ alkyl, C₅ alkyl, or C₆ alkyl; unsubstituted aryl or substituted aryl, including unsubstituted phenyl, or phenyl having 1, 2, 3, 4, or 5 substituents; unsubstituted heteroaryl or substituted heteroaryl, including unsubstituted pyridine or pyridine having 1, 2, 3, or 4 substituents, or unsubstituted pyrimidine or pyrimidine having 1, 2, or 3 substituents. Any substituent may be used in the substituted aryl (e.g., substituted phenyl) or substituted heteroaryl (e.g., substituted pyridine or substituted pyrimidine). For example, the substituents of the substituted aryl or substituted heteroaryl may independently be, halo (such as F, Cl, Br, I), C₁₋₆ alkyl (such as CH₃, C₂ alkyl, C₃ alkyl, C₄ alkyl, C₅ alkyl, C₆ alkyl), or X-Ra, wherein X is O, —C(═O)—, —NHC(═O)—, or —C(═O)NH—, and Ra is C₁₋₆ alkyl (such as CH₃, C₂ alkyl, C₃ alkyl, C₄ alkyl, C₅ alkyl, C₆ alkyl), C₂₋₆ alkenyl (such as —CH═CH₂, —CH₂CH═CH₂, —CH₂CH₂CH═CH₂, —CH₂CH₂CH₂CH═CH₂, —CH₂CH₂CH₂CH₂CH═CH₂, etc.), or C₂₋₆ alkynyl (such as —CH≡CH₂, —CH₂CH≡CH₂, —CH₂CH₂CH≡CH₂, —CH₂CH₂CH₂CH≡CH₂, —CH₂CH₂CH₂CH₂CH≡CH₂, etc.); or azide.

In some embodiments, R₄ and R₅ are the same functional group.

In some embodiments, R₄ and R₅ are different functional groups.

In some embodiments, R₄ is C₁₋₆ alkyl, such as methyl, and R₅ is optionally substituted phenyl.

In some embodiments, R₄ is C₁₋₆ alkyl, such as methyl, and R₅ is optionally substituted pyridine.

In some embodiments, R₄ is C₁₋₆ alkyl, such as methyl, and R₅ is optionally substituted pyrimidine.

In some embodiments, R₄ is optionally substituted pyridine and R₅ is optionally substituted pyridine.

In some embodiments, R₄ is optionally substituted phenyl and R₅ is optionally substituted phenyl.

In some embodiments, R₄ is optionally substituted phenyl and R₅ is optionally substituted pyrimidine.

In some embodiments, R is an optionally substituted phenoxazine or an optionally substituted phenothiazine, such as phenoxazine or phenothiazine having the formula:

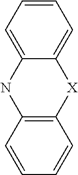

which may additionally have substituents on any available ring position.

X is selected from: O and S.

R is attached to the azacycle via R's nitrogen.

Substituents of R may independently include halogen, alkyl (e.g., C₁₋₆ alkyl, such as CH₃, C₂ alkyl, C₃ alkyl, C₄ alkyl, C₅ alkyl, or C₆ alkyl), alkoxy (e.g., C₁₋₆ alkoxy, such as —OCH₃, C₂ alkoxy, C₃ alkoxy, C₄ alkoxy, C₅ alkoxy, or C₆ alkoxy), N₃, NO₂, and CN.

It will be understood that compounds described herein may exist as stereoisomers, enantiomers, diastereomers, cis, trans, syn, anti, solvates (including hydrates), tautomers, and mixtures thereof.

In certain embodiments, an antiviral compound is compound 325, having the formula:

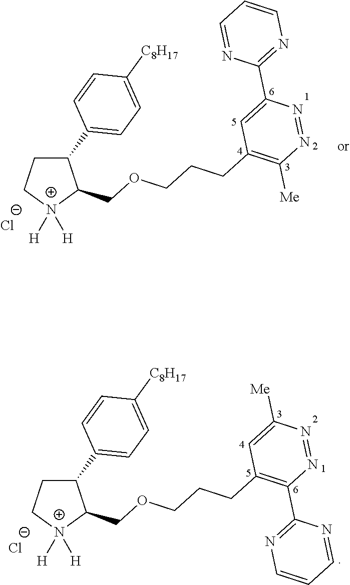

### D. Sphingolipid-Like Compounds Based on 2-C-aryl Azacycles

In certain embodiments, an antiviral compound is based on diastereomeric 2-C-aryl azacycles. In certain embodiments, an antiviral compound is of formula:

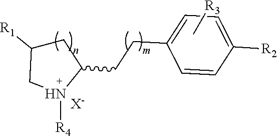

R₁ is a functional group selected from H, an alkyl chain, OH, (CH₂)ₙOH, CHOH-alkyl, CHOH-alkyne, (CH₂)ₙOR′, where R′ is an alkyl, alkene or alkyne.

R₂ is an aliphatic chain (C₆-C₁₄).

R₃ is a mono-, di-, tri- or tetra-aromatic substituent that includes hydrogen, halogen, alkyl, alkoxy, azide (N₃), ether, NO₂, cyanide (CN), or a combination thereof.

R₄ is a functional group selected from H, alkyl including methyl (Me), ester, or acyl.

X⁻ is an anion of the suitable acid.

n is an independently selected integer selected from 1, 2, or 3.

m is an independently selected integer selected from 0, 1 or 2.

The molecule can include an optional functional group of the azacycle's substituent selected from the following:
a polar group in the alpha, beta or gamma position with regard to the azacycle selected from carbonyls (C═O) and alcohols (CHOH);
a cyclic carbon chain extending from the alpha, beta or gamma positions with regard to the azacycle back to the N of the azacycle, and
a combination thereof.

In some embodiments, R₁ is H, OH, or CH₂OH. In some embodiments, R₁ is H. In some embodiments, R₁ is OH. In some embodiments, R₁ is CH₂OH.

In some embodiments, R₂ is C₆₋₁₄ alkyl, C₆₋₁₀ alkyl, C₇₋₉ alkyl, C₆H₁₃, C₇H₁₅, C₈H₁₇, C₃H₁₃, C₁₀H₂₁, C₁₁H₂₃, C₁₂H₂₅, C₁₃H₂₇, or C₁₄H₂₉. In some embodiments, R₂ is C₈H₁₇.

In some embodiments R₃ is H.

In some embodiments, n is 1.

In some embodiments m is 0. In some embodiments, m is 1. In some embodiments, m is 2. In some embodiments, m is 3.

In some embodiments, the linking group connecting the phenyl ring to the azacycle is C(═O), CH₂C(═O), C(═O)CH₂, CH₂CH₂C(═O), CH₂, CH₂CH₂, CH₂C(OCH₃)H, or CHOHCH₂. In some embodiments, the linking group connecting the phenyl ring to the azacycle is C(═O). In some embodiments, the linking group connecting the phenyl ring to the azacycle is CH₂C(═O). In some embodiments, the linking group connecting the phenyl ring to the azacycle is C(═O)CH₂. In some embodiments, the linking group connecting the phenyl ring to the azacycle is CH₂CH₂C(═O). In some embodiments, the linking group connecting the phenyl ring to the azacycle is CH₂. In some embodiments, the linking group connecting the phenyl ring to the azacycle is CH₂CH₂. In some embodiments, the linking group connecting the phenyl ring to the azacycle is CH₂C(OCH₃)H. In some embodiments, the linking group connecting the phenyl ring to the azacycle is CHOHCH₂.

In some embodiments, the linking group connecting the phenyl ring to the azacycle includes a cyclic carbon chain extending from the alpha, beta or gamma positions with regard to the azacycle back to the N of the azacycle, so that the azacycle with the linking group form an optionally substituted bicyclic ring of the formula:

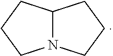

In some embodiments, R₄ is H. In some embodiments, R₄ is C₁₋₆ alkyl, such as CH₃, C₂H₅, C₃H₇, C₄H₉, C₅H₁₁, C₆H₁₃, C₁₋₃ alkyl, etc., C₁₋₆ acyl, or C₁₋₆ ester. In some embodiments, R₄ is methyl.

In still other embodiments, the R₂ and R₃ substituents can have different combinations around the phenyl ring with regard to their position.

In still other embodiments, R₂ is an unsaturated hydrocarbon chain.

In still other embodiments, the R₁ is an alkyl having 1 to 6 carbons.

It will be understood that compounds described herein may exist as stereoisomers, enantiomers, diastereomers, cis, trans, syn, anti, solvates (including hydrates), tautomers, and mixtures thereof.

### E. Pharmaceutical Salts of Sphingolipid-Like Compounds

Certain sphingolipid-like compounds can also be related to pharmaceutically acceptable salts. A “pharmaceutically acceptable salt” retains the desirable biological activity of the compound without undesired toxicological effects. Salts can be salts with a suitable acid, including, but not limited to, hydrochloric acid, hydrobromic acid, sulfuric acid, phosphoric acid, nitric acid and the like; acetic acid, oxalic acid, tartaric acid, succinic acid, malic acid, benzoic acid, pamoic acid, alginic acid, methanesulfonic acid, naphthalenesulphonic acid, and the like. Also, incorporated cations can include ammonium, sodium, potassium, lithium, zinc, copper, barium, bismuth, calcium, and the like; or organic cations such as tetraalkylammonium and trialkylammonium cations. Also useful are combinations of acidic and cationic salts. Included are salts of other acids and/or cations, such as salts with trifluoroacetic acid, chloroacetic acid, and trichloroacetic acid.

### Certain Pharmaceutical Compositions

In certain embodiments, the present disclosure provides pharmaceutical compositions comprising one or more antiviral compounds or a salt thereof. In certain such embodiments, the pharmaceutical composition comprises a suitable pharmaceutically acceptable diluent or carrier. In certain embodiments, a pharmaceutical composition comprises a sterile saline solution and one or more antiviral compounds. In certain embodiments, the sterile saline is pharmaceutical grade saline. In certain embodiments, a pharmaceutical composition comprises sterile water and one or more antiviral compounds. In certain embodiments, the water is pharmaceutical grade water. In certain embodiments, a pharmaceutical composition comprises phosphate-buffered saline (PBS) and one or more antiviral compounds. In certain embodiments, the PBS is pharmaceutical grade PBS.

In certain embodiments, pharmaceutical compositions comprise one or more antiviral compounds and one or more excipients. In certain such embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, antiviral compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an antiviral compound encompass any pharmaceutically acceptable salts of the antiviral compound, esters of the antiviral compound, or salts of such esters. In certain embodiments, pharmaceutical compositions comprising antiviral compound encompass any pharmaceutically acceptable salts of the antiviral compound. In certain embodiments, pharmaceutical compositions comprising one or more antiviral compounds, upon administration to an animal, including a human, are capable of providing (directly or indirectly) the biologically active metabolite or residue thereof. Accordingly, for example, the disclosure is also drawn to pharmaceutically acceptable salts of antiviral compounds, prodrugs, pharmaceutically acceptable salts of such prodrugs, and other bioequivalents. Suitable pharmaceutically acceptable salts include, but are not limited to, sodium and potassium salts. In certain embodiments, prodrugs comprise one or more conjugate group attached to an antiviral compound, wherein the conjugate group is cleaved by endogenous nucleases within the body.

In certain embodiments, a pharmaceutical composition comprises a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethyl sulfoxide (DMSO) are used.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose. In certain embodiments, dimethyl sulfoxide (DMSO) is utilized as a co-solvent. In certain embodiments, cremophor (or cremophor EL) is utilized as a co-solvent.

In certain embodiments, pharmaceutical compositions comprise one or more compounds that increase bioavailability. For example, 2-hydroxypropyl-beta-cyclodextrin can be utilized in pharmaceutical compositions and may increase bioavailability. In certain embodiment, DMSO, cremophor and 2-hydroxypropyl-beta-cyclodextrin, and mixtures thereof, may be utilized to increase bioavailability of various antiviral compounds, especially sphingolipid-like compounds.

In certain embodiments, pharmaceutical compositions are prepared for oral administration. In certain embodiments, pharmaceutical compositions are prepared for buccal administration. In certain embodiments, pharmaceutical compositions are prepared for aerosol or nebulizer administration. In certain embodiments, a pharmaceutical composition is prepared for administration by injection (e.g., intravenous, subcutaneous, intramuscular, etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

In certain embodiments, a pharmaceutical composition is administered in a therapeutically effective amount as part of a course of treatment. As used in this context, to “treat” means to ameliorate or prevent at least one symptom of the disorder to be treated or to provide a beneficial physiological effect. A therapeutically effective amount can be an amount sufficient to prevent reduce, ameliorate or eliminate the symptoms of diseases or pathological conditions susceptible to such treatment. In certain embodiments, a therapeutically effective amount is an amount sufficient to inhibit virus replication.

Dosage, toxicity and therapeutic efficacy of a pharmaceutical composition can be determined, e.g., by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Compounds that exhibit high therapeutic indices are preferred. While compounds that exhibit toxic side effects may be used, care should be taken to design a delivery system that targets such compounds to the site of affected tissue in order to minimize potential damage to uninfected cells and, thereby, reduce side effects.

Data obtained from cell culture assays or animal studies can be used in formulating a range of dosage for use in humans. If a pharmaceutical composition is provided systemically, the dosage of such compounds lies preferably within a range of circulating concentrations that include the ED₅₀ with little or no toxicity. The dosage may vary within this range depending upon the dosage form employed and the route of administration utilized. For any compound used in a method described herein, the therapeutically effective dose can be estimated initially from cell culture assays. A dose may be formulated in animal models to achieve a circulating plasma concentration or within the local environment to be treated in a range that includes the IC₅₀ (i.e., the concentration of the test compound that achieves a half-maximal inhibition of virus propagation) as determined in cell culture. Such information can be used to more accurately determine useful doses in humans. Levels in plasma may be measured, for example, by liquid chromatography coupled to mass spectrometry.

An “effective amount” is an amount sufficient to effect beneficial or desired results. For example, a therapeutic amount is one that achieves the desired therapeutic effect. This amount can be the same or different from a prophylactically effective amount, which is an amount necessary to prevent onset of disease or disease symptoms. An effective amount can be administered in one or more administrations, applications or dosages. A therapeutically effective amount of a composition depends on the composition selected. The compositions can be administered one from one or more times per day to one or more times per week; including once every other day. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of a pharmaceutical composition described herein can include a single treatment or a series of treatments. For example, several divided doses may be administered daily, one dose, or cyclic administration of the compounds to achieve the desired therapeutic result. A single small molecule compound may be administered, or combinations of various small molecule compounds may also be administered.

It is also possible to add agents that improve the solubility of pharmaceutical compositions. For example, a pharmaceutical composition can be formulated with one or more adjuvants and/or pharmaceutically acceptable carriers according to the selected route of administration. For oral applications, gelatin, flavoring agents, or coating material can be added. In general, for solutions or emulsions, carriers may include aqueous or alcoholic/aqueous solutions, emulsions or suspensions, including saline and buffered media. Parenteral vehicles can include sodium chloride and potassium chloride, among others. In addition, intravenous vehicles can include fluid and nutrient replenishers, electrolyte replenishers and the like.

Numerous coating agents can be used in accordance with various embodiments. In certain embodiments, the coating agent is one which acts as a coating agent in conventional delayed release oral formulations, including polymers for enteric coating. Examples include hypromellose phthalate (hydroxy propyl methyl cellulose phthalate; HPMCP); hydroxypropylcellulose (HPC; such as KLUCEL®); ethylcellulose (such as ETHOCEL®); and methacrylic acid and methyl methacrylate (MAA/MMA; such as EUDRAGIT®).

In certain embodiments, a pharmaceutical composition also includes at least one disintegrating agent, as well as diluent. In some embodiments, a disintegrating agent is a super disintegrant agent. One example of a diluent is a bulking agent such as a polyalcohol. In many embodiments, bulking agents and disintegrants are combined, such as, for example, PEARLITOL FLASH®, which is a ready to use mixture of mannitol and maize starch (mannitol/maize starch). In accordance with a number of embodiments, any polyalcohol bulking agent can be used when coupled with a disintegrant or a super disintegrant agent. Additional disintegrating agents include, but are not limited to, agar, calcium carbonate, maize starch, potato starch, tapioca starch, alginic acid, alginates, certain silicates, and sodium carbonate. Suitable super disintegrating agents include, but are not limited to crospovidone, croscarmellose sodium, AMBERLITE (Rohm and Haas, Philadelphia, Pa.), and sodium starch glycolate.

In certain embodiments, diluents are selected from the group consisting of mannitol powder, spray dried mannitol, microcrystalline cellulose, lactose, dicalcium phosphate, tricalcium phosphate, starch, pregelatinized starch, compressible sugars, silicified microcrystalline cellulose, and calcium carbonate.

In certain embodiments, a pharmaceutical composition further utilizes other components and excipients. For example, sweeteners, flavors, buffering agents, and flavor enhancers to make the dosage form more palatable. Sweeteners include, but are not limited to, fructose, sucrose, glucose, maltose, mannose, galactose, lactose, sucralose, saccharin, aspartame, acesulfame K, and neotame. Common flavoring agents and flavor enhancers that may be included in the formulations described herein include, but are not limited to, maltol, vanillin, ethyl vanillin, menthol, citric acid, fumaric acid, ethyl maltol and tartaric acid.

In certain embodiments, a pharmaceutical composition also includes a surfactant. In certain embodiments, surfactants are selected from the group consisting of Tween 80, sodium lauryl sulfate, and docusate sodium.

In certain embodiments, a pharmaceutical composition further utilizes a binder. In certain embodiments, binders are selected from the group consisting of povidone (PVP) K29/32, hydroxypropylcellulose (HPC), hydroxypropylmethylcellulose (HPMC), ethylcellulose (EC), corn starch, pregelatinized starch, gelatin, and sugar.

In certain embodiments, a pharmaceutical composition also includes a lubricant. In certain embodiments, lubricants are selected from the group consisting of magnesium stearate, stearic acid, sodium stearyl fumarate, calcium stearate, hydrogenated vegetable oil, mineral oil, polyethylene glycol, polyethylene glycol 4000-6000, talc, and glyceryl behenate.

Preservatives and other additives, like antimicrobial, antioxidant, chelating agents, and inert gases, can also be present. (See generally, Remington's Pharmaceutical Sciences, 16th Edition, Mack, (1980), the disclosure of which is incorporated herein by reference.)

### Modes of Treatments

In certain embodiments, viral compounds are administered in a therapeutically effective amount as part of a course of treatment. As used in this context, to “treat” means to ameliorate or prevent at least one symptom of the viral infection to be treated or to provide a beneficial physiological effect. For example, one such amelioration of a symptom could be inhibition of virus replication. Assessment of virus replication can be performed in many ways, including, but not limited to assessing viral genome load (e.g., PCR test), detection of viral markers (e.g., antigen detection test), or performance of viral plaque assay.

A number of embodiments are directed towards treating an individual for a viral infection or prophylactically inhibiting viral infection in people at high risk of infection. Accordingly, an embodiment to treat or prophylactically inhibiting infection of an individual is as follows:
(i) diagnose or determine that an individual has a viral infection of an enveloped virus or at risk of infection with an enveloped virus;
(ii) administer to the individual an antiviral compound that agonizes PP2A.

A number of embodiments are directed towards prophylactically treating an individual for a viral infection. Accordingly, an embodiment to treat an individual is as follows:
(i) identify an individual for prophylactic treatment (e.g., an individual traveling to an endemic region, an individual at risk of exposure, and individual at risk of severe sickness)
(ii) administer to the individual an antiviral compound that agonizes PP2A

In certain embodiments, an individual to be treated has been diagnosed as having a viral infection. In certain embodiments, the viral infection is of an enveloped virus. A number of infections of enveloped virus can be treated, including (but not limited to) infections of coronavirus (e.g., severe acute respiratory syndrome corona virus 2 (SARS-CoV-2)), herpesvirus (e.g., chicken pox), poxvirus (e.g., small pox), retrovirus (e.g, human immunodeficiency virus (HIV)), flavivirus (e.g., dengue virus, zika virus), hepadnavirus (e.g., hepatitis B), pneumovirus (e.g., respiratory syncytial virus (RSV)), influenza virus, ebolavirus, rabies virus, mumps virus, and papillomavirus.

A therapeutically effective amount can be an amount sufficient to prevent, reduce, ameliorate or eliminate the symptoms of diseases or pathological conditions susceptible to such treatment, such as, for example, viral infections of enveloped viruses. In certain embodiments, a therapeutically effective amount is an amount sufficient to reduce viral replication, hijacking of host cellular machinery, inhibition of a host's antiviral response, syncytia, and/or virulence.

### EXEMPLARY EMBODIMENTS

The following examples illustrate certain embodiments of the present disclosure and are not limiting. Biological data supports the use of the antiviral compounds in accordance of a variety of embodiments for use within a treatment of viral infections. It is noted that embodiments of the compounds described herein, in accordance with the disclosure, mitigate and/or inhibit enveloped virus activity. Accordingly, embodiments using these compounds to treat various diseases, such as COVID-19 and influenza.

### Example 1: Effects of Sphingolipid-Like Compounds Treatment on Virus Activity

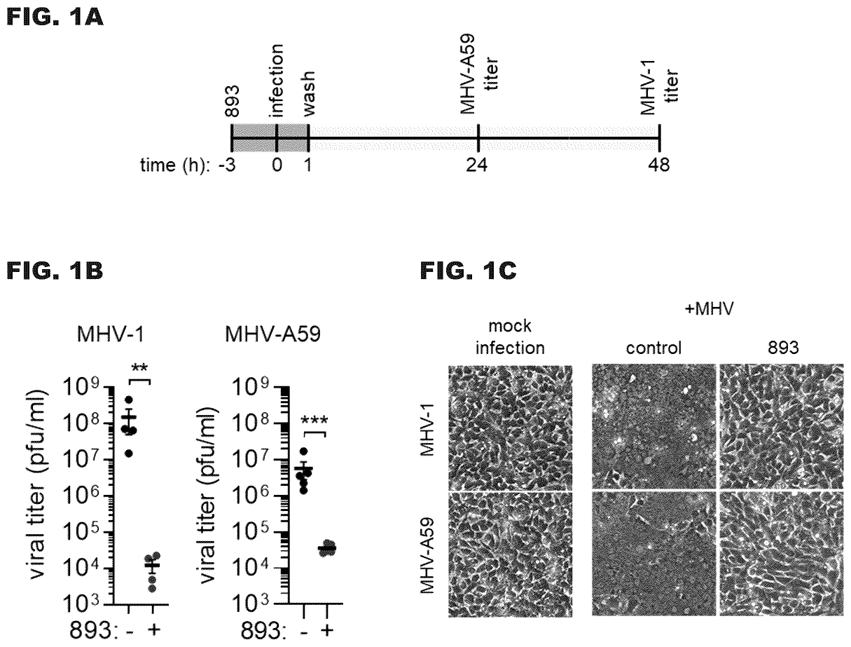

To determine whether PP2A activation disrupts coronavirus infection and early replication, a class of coronaviruses called murine hepatitis virus (MHV), which shares the same replication cycle as other coronaviruses, including SARS-CoV-2, SARS-CoV, and MERS-CoV, was treated with sphingolipid-like compounds. To test the efficacy of compound 893, murine astrocytoma cells (DBT cells) were treated with 4-5 μM of compound 893 for three hours prior to infection with MHV-1 or MHV-A59 at a multiplicity of infection (MOI) of 0.1. Cells were washed 1 hour after infection, and then viral titers measured by plaque assay at 24 hours or 48 hours, depending on virus (FIG. 1A). Indeed, activating PP2A with 893 just prior to and during infection reduced viral titers of MHV-1 and MHV-A59 by 3-4 logs (FIGS. 1B and 1C). The results suggest that compound 893 blocks viral entry and early replication.

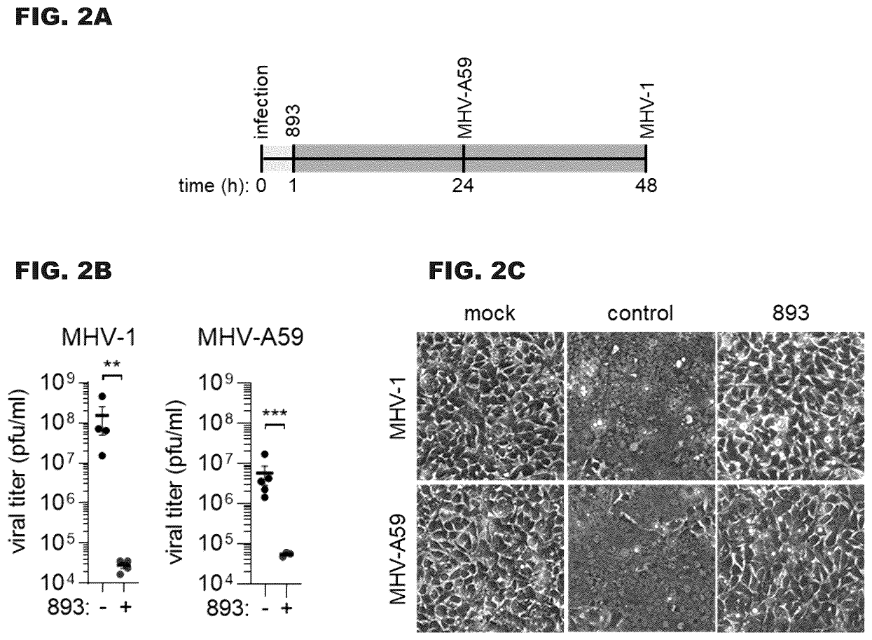

In another experiment, PP2A activation was assessed for its ability to disrupt MHV replication and viral release. DBT cells were first infected with MHV-1 or MHV-A59 at a multiplicity of infection (MOI) of 0.1 and one hour later treated with 4-5 μM of compound 893. Cells were left growing in the presence of virus and the 893 compound, and then viral titers measured by plaque assay at 24 hours or 48 hours, depending on viral strain (FIG. 2A). Indeed, activating PP2A with 893 after infection reduced viral titers of MHV-1 and MHV-A59 by 3-4 logs (FIGS. 2B and 2C). The results suggest that compound 893 blocks viral replication and assembly/exit.

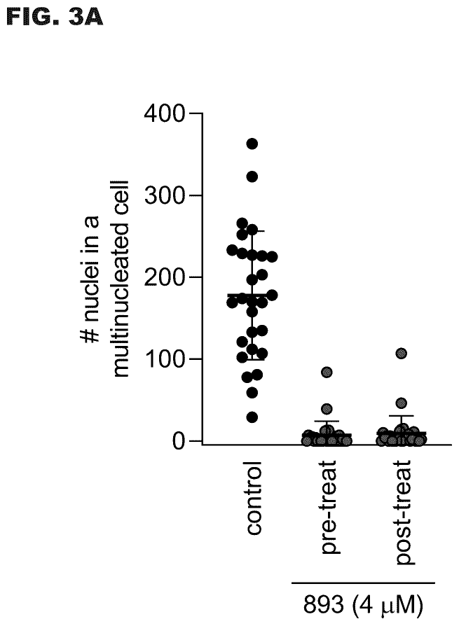

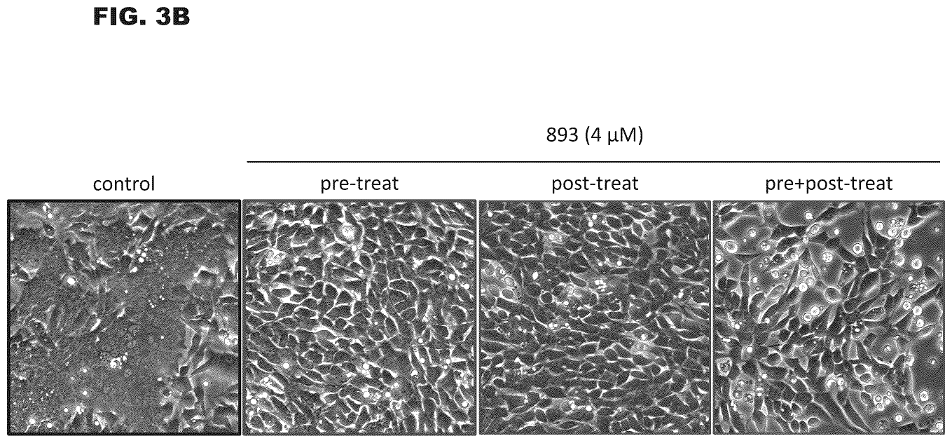

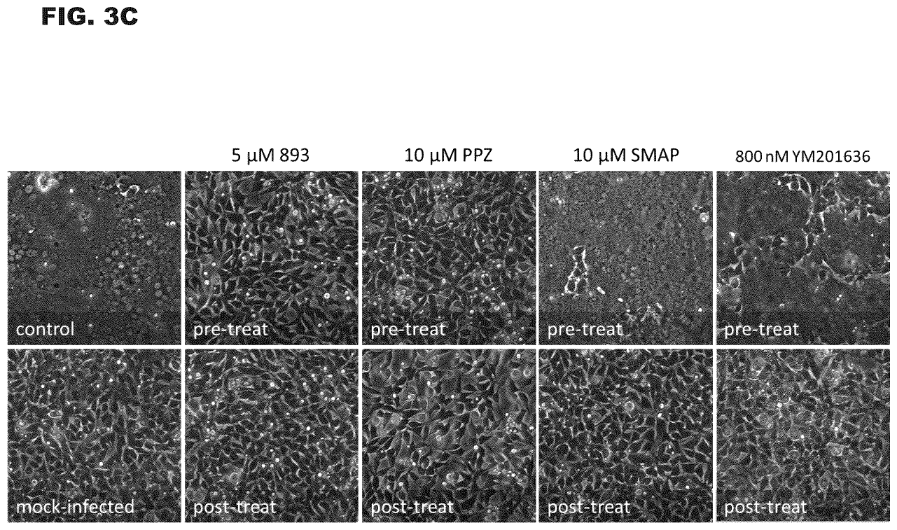

To determine whether PP2A activation mitigates syncytium formation induced by coronaviruses, DBT cells were treated with 4 μM of compound 893 before or after infection with MHV-A59 at a multiplicity of infection (MOI) of 0.1 and then assessed for syncytium formation via phase contrast microscopy 24 hours post infection. Compound 893 potently prevented syncytium formation induced by MHV-A59 (FIGS. 3A and 3B) or MHV-1 (FIG. 3C) infection when used as a pre-treatment or after viral infection (post-treatment). These results suggest that trafficking of the viral spike protein and/or the viral receptor to the cell surface was blocked by PP2A activation.

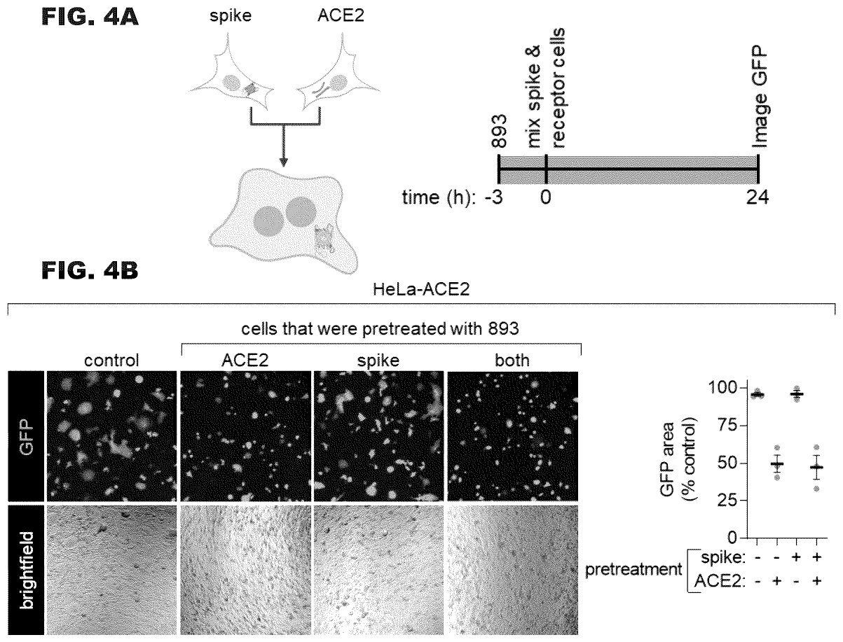

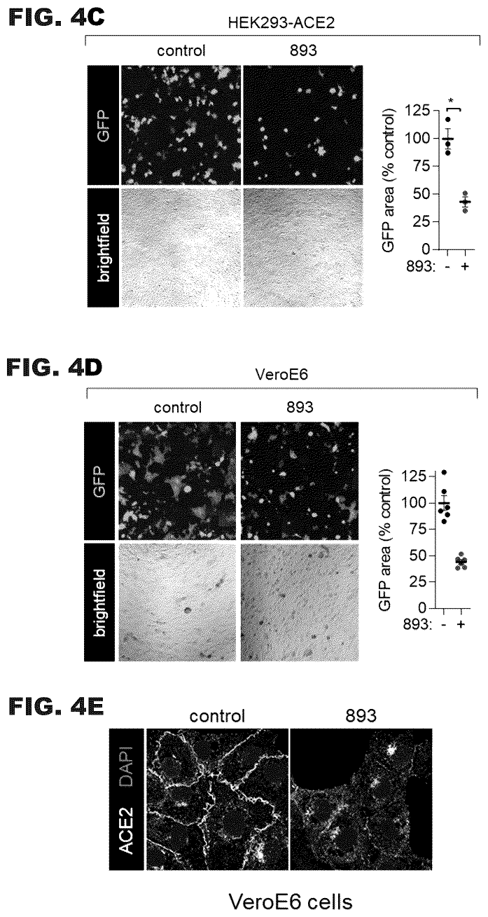

It was also assessed whether compound 893 mitigates syncytium formation induced by expression of the SARS-CoV-2 viral spike protein. To perform this assessment, various cell lines expressing SARS-CoV-2 spike or the ACE2 receptor were mixed and assays measuring syncytium formation performed. Effector cells were transfected with the spike protein and the N-terminal fragment of GFP and the target cells were transfected with the receptor ACE2 and the C-terminal fragment of GFP. ACE2 is the host receptor of SARS-CoV-2 and interacts with the SARS-CoV-2 S protein to promote endocytosis of the virus. Because each cell is transfected with a partial GFP protein, none of the cells fluoresce after transfection. Syncytium formation and fusion between a cell with the N-terminal fragment of GFP and a cell with C-terminal fragment is required to form a functional GFP product capable of fluorescence. In this experiment, four sets pre-treatments with 10 μM of compound 893 were performed:
(1) Control: no pre-treatment of spike expressing or ACE2 expressing cells
(2) Pre-treatment of ACE2 expressing cells only
(3) Pre-treatment of spike expressing cells only
(4) Pre-treatment of both ACE2 expressing and spike expressing cells (FIG. 4A).
   After three hours of pre-treatment, the spike expressing and ACE2 expressing cells were mixed and area occupied by GFP-positive cells (syncytia) assessed. Experimental results for HeLa cells (FIG. 4B), HEK293 cells (FIG. 4C) and VeroE6 cells (FIG. 4D) show that pre-treatment with compound 893 reduced the amount GFP area, signifying a reduction of syncytium formation and fusion when both ACE2 and spike expressing cells were pre-treated with 893. As treating only spike expressing cells was not effective (FIG. 4A), these results suggest a reduction of ACE2 receptor localization at the outer cell membrane, which was shown in pre-treated VeroE6 cells (FIG. 4E).

### Example 2: Effects of PP2A Agonists, PIKfyve Antagonists, and ARF6 Antagonists

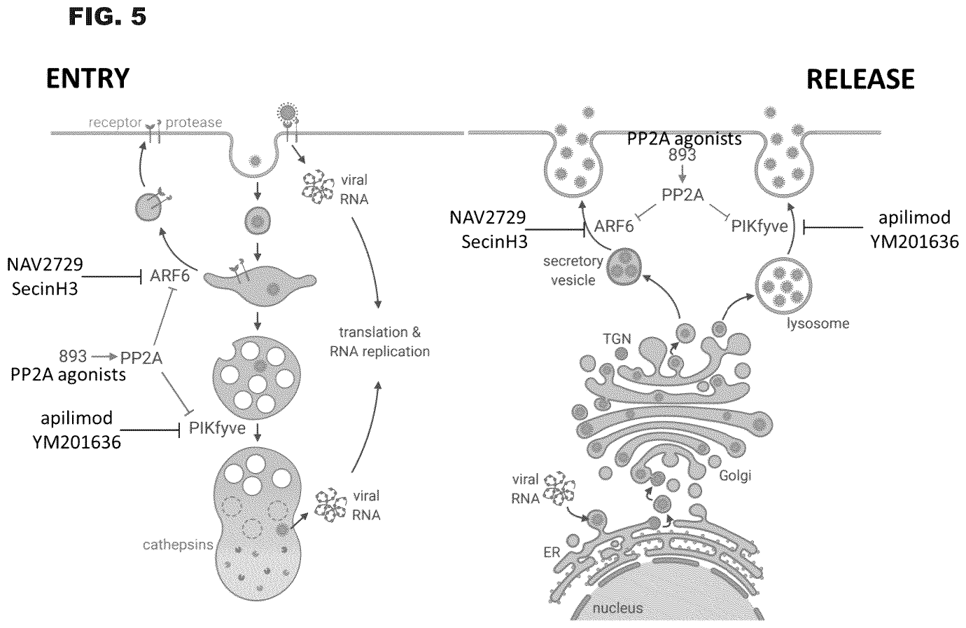

Utilizing the endocytic and exocytic pathway, enveloped virus are taken up into cells, where they replicate to make more genomes and viral capsids, and eventually exit as mature virions. Once a virus comes into contact with a cell's membrane, the viral spike/envelope proteins can interact with activating proteases (e.g. TMPRSS2) and cell surface receptors (e.g., coronavirus interacts with the ACE2 receptor), leading to membrane fusion and internalization of the virus (FIG. 5). There are several factors, however, that can reduce enveloped virus entry and replication. For instance, Phosphoinositide Kinase, FYVE-Type Zinc Finger Containing (PIKfyve) is one enzyme that promotes release of the viral contents into cytosol for replication and release of mature virions into the extracellular space promoting a spreading infection (FIG. 5). In addition, the cytosolic enzyme ADP Ribosylation Factor 6 (ARF6) can promote the both the surface expression of viral receptors and activating proteases and the fusion of virion-containing endocytic vesicles with the plasma membrane (FIG. 5). Here, the present disclosure provides compounds and methods of mitigating enveloped viral entry and replication through blocking or slowing endosome function. In certain embodiments, viral content release into the cytosol is reduced by utilizing PIKfyve antagonists. In certain embodiments, viral entry and mature virus release is reduced by utilizing ARF6 antagonists. As explained in this Example, it is now known that protein phosphatase 2 (PP2A) agonists inhibit viral entry and the release of mature virions. Accordingly, in certain embodiments, PP2A agonists are utilized to mitigate virus activity in a cell.

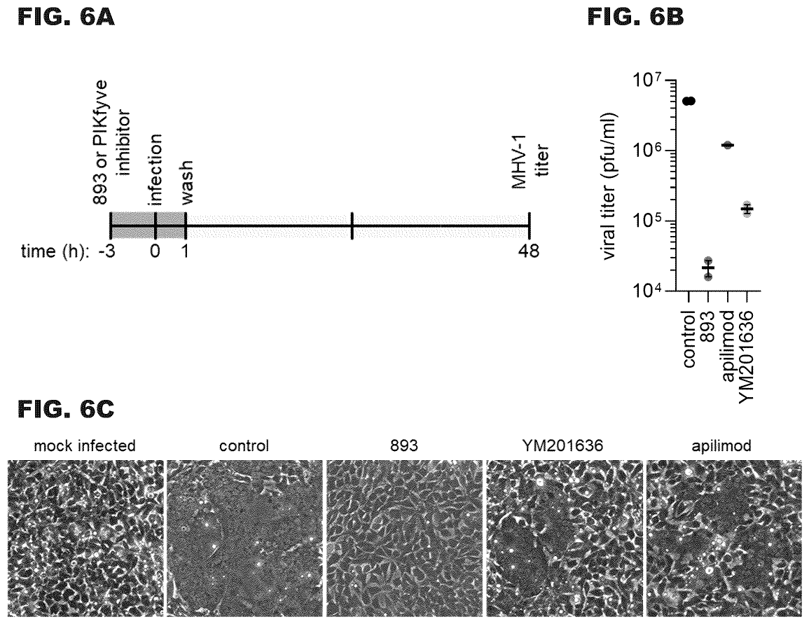

To determine the effect of PIKfyve inhibition on enveloped virus propagation, DBT cells were pre-treated with one of the PIKfyve inhibitors (400 nM of YM201636 or 50 nM of apilimod) or compound 893 (10 μM) for three hours, infected with MHV-1, and then washed and fresh medium added after 1 hour (FIG. 6A). Control cells were not pre-treated with any compound. Forty-eight hours post infection, viral titers of MHV-1 were assessed using a plaque assay. Compound 893 reduced viral titers nearly 3-logs whereas the PIKfyve inhibitors reduced titers less than 2-logs (FIG. 6B). Images of the cells acquired at 48 hours show the inhibitory effect of the compounds on syncytium formation and fusion which is greater for 893 than for YM201636 or apilimod (FIG. 6C).

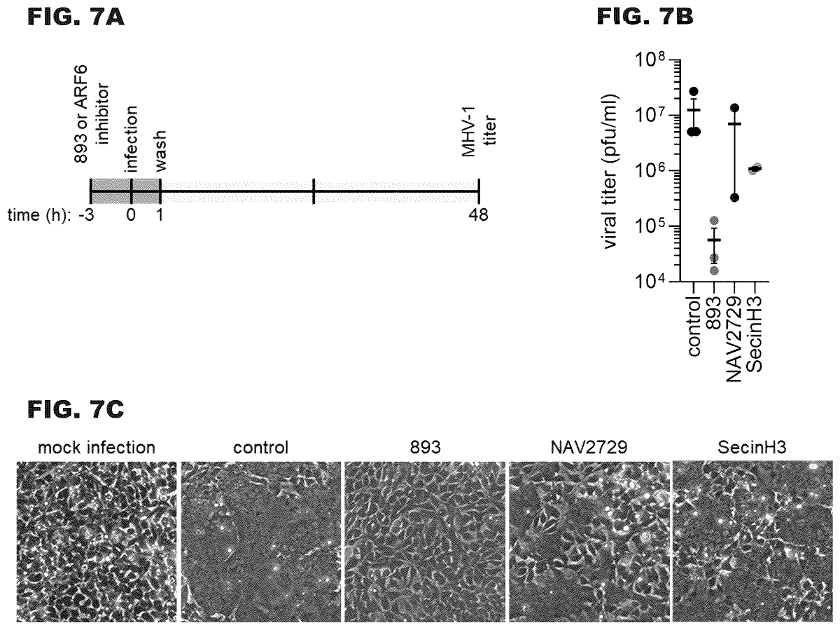

To determine the effect of ARF6 inhibition on enveloped virus replication and release, DBT cells were pre-treated with one of the ARF6 inhibitors (5 μM of NAV2729 or 30 μM of SecinH3) or compound 893 (10 μM) for three hours, infected with MHV-1 (FIG. 7A), and then washed 1 hour later when fresh medium was added. Control cells were not pre-treated with any compound. Forty-eight hours post infection, viral titers of MHV-1 were assessed using a plaque assay. Compound 893 reduced viral titers nearly 3-logs whereas the ARF6 inhibitors reduced titers about 1-log (FIG. 7B). Images of the cells were acquired at 48 hour and show the inhibitory effect of the compounds on syncytium formation and cell-cell fusion (FIG. 7C).

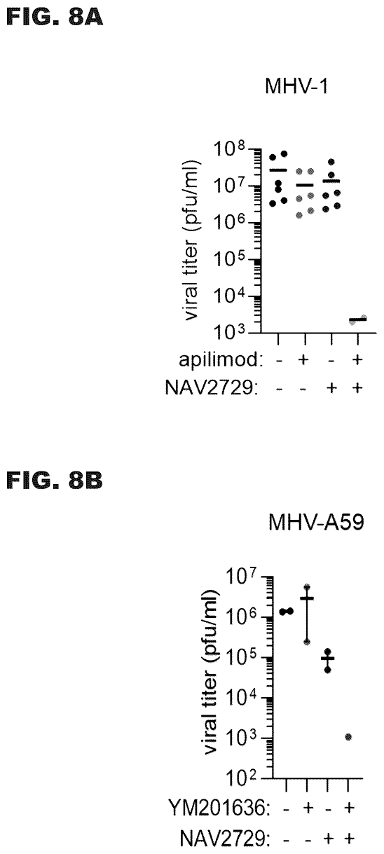

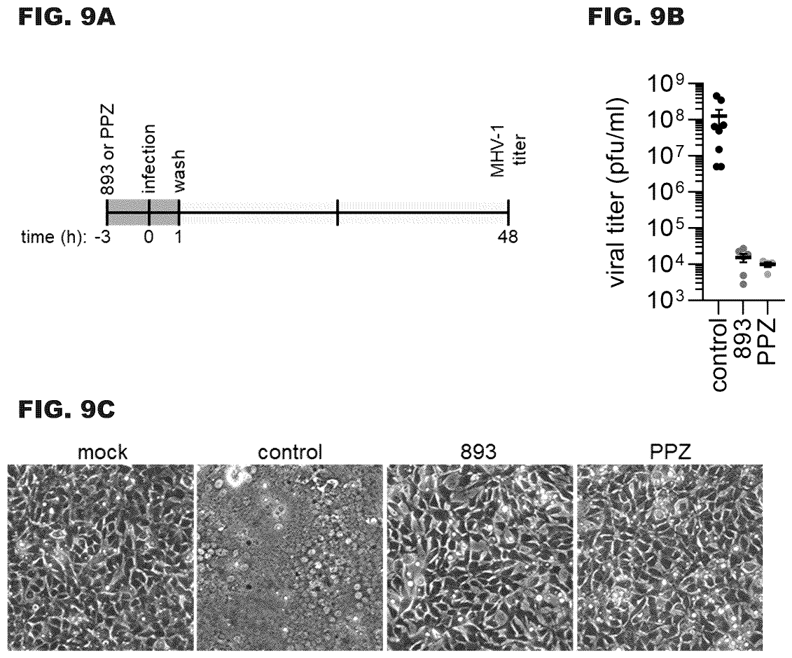

Interestingly, it was found that combining a PIKfyve inhibitor with an ARF6 inhibitor reduced viral titers of MHV-1 or MHV-A59 similar to compound 893 (FIGS. 8A and 8B). These results suggest that inhibiting both PIKfyve and ARF6 provides a better means for disrupting enveloped viral activity than either inhibitor alone. Because PP2A agonists (such as sphingolipid-like compounds) inhibit both PIKfyve and ARF6, these agonists would be more useful for treating infections with enveloped viruses. Indeed, the PP2A agonist perphenazine (PPZ) reduced viral titers of MHV-1 more effectively than either a PIKfyve inhibitor or ARF6 inhibitor alone and to a degree similar to that of compound 893 (FIGS. 9A and 9B). PPZ also reduced syncytium formation and fusion as efficiently as compound 893 (FIG. 9C).

### Example 3: Compound 893 Pre-Clinical Data

Sphingolipid-like compound 893 is water-soluble, orally bioavailable, resistant to metabolism, and has a 10.6 h half-life upon oral administration. Studies show that 5 daily doses of 120 mg/kg P.O. yield>100 μM compound 893 many tissues relevant to viral infection, including the lungs, brain, and liver, key sites of coronavirus replication. Only 1-10 μM 893 is required to inhibit endolysosomal trafficking in vitro, but total drug levels are 300-fold higher in the lungs likely because compound 893 is highly bound by serum proteins and held inactive.

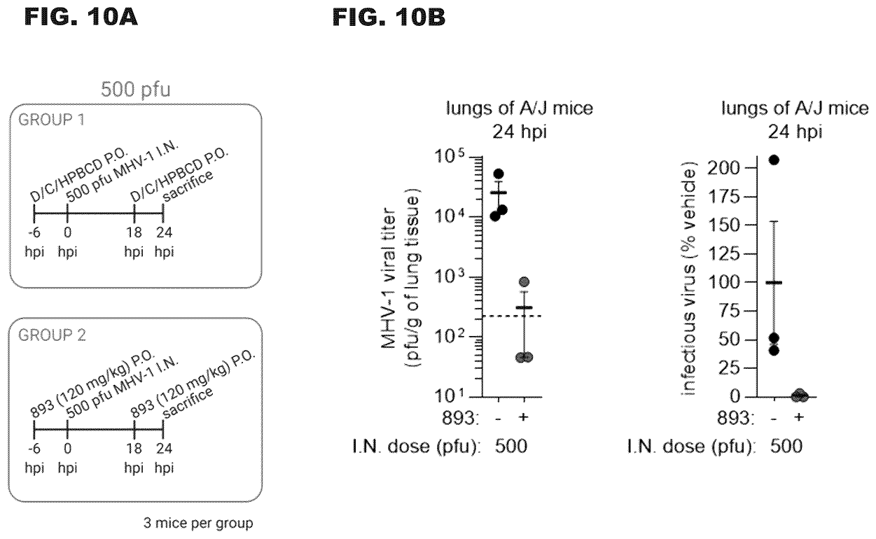

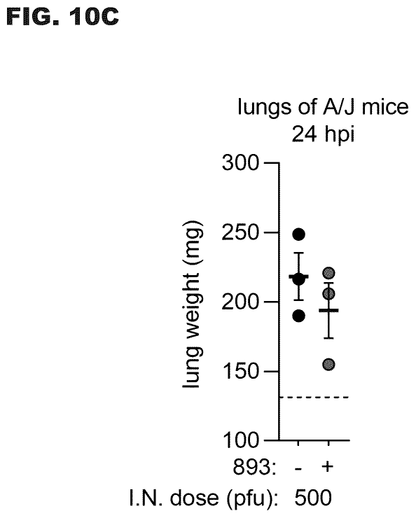

An experiment was performed using male A/J mice (6-8 weeks old) to determine the ability of compound 893 to reduce viral activity in vivo. Six hours prior to infection with MHV-1 virus, the mice were pre-treated by gavage with vehicle (5% DMSO, 5% cremophor EL, 18% HPBCD) or 120 mg/kg of compound 893 in vehicle. Then, the mice were infected intranasally with 500 pfu of MHV-1 and then re-treated with vehicle only or compound 893 with vehicle 18 hours after infection. Mice were sacrificed at 24 hours post-infection and their lungs were assessed for viral titer and weight (lung weight increases with inflammation and some effective COVID-19 treatments such as glucocorticoids work by reducing inflammation). Mice treated with compound 893 had a 2-log reduction in infectious virus within the lungs (FIG. 10B) and exhibited a reduced lung weight (FIG. 10C). These results suggest that an orally administered PP2A agonist can reduce viral activity in vivo and would be useful in the treatment of an enveloped virus, such as coronaviruses.

### DOCTRINE OF EQUIVALENTS

While the above description contains many specific embodiments of the invention, these should not be construed as limitations on the scope of the invention, but rather as an example of one embodiment thereof. Accordingly, the scope of the invention should be determined not by the embodiments illustrated, but by the appended claims and their equivalents.

What is claimed is:

1. A method of mitigating viral activity in a biological cell, comprising:
contacting a biological cell with one or more antiviral compounds,
wherein the one or more antiviral compounds comprise an agonist of protein phosphatase 2 (PP2A) or an antagonist of ADP Ribosylation Factor 6 (ARF6), and
wherein the biological cell is infected with an enveloped virus or at risk of being infected with an enveloped virus.

2. The method as in claim 1, wherein the one or more antiviral compounds comprise further comprise an antagonist of PIKfyve.

3. The method as in claim 1, wherein the PP2A is agonist is: a sphingolipid, a sphingolipid-like compound, perphenazine, a perphenazine derivative, a SET inhibitor, a CIP2a inhibitor, Withaferin A, OSU-2S, FTY720, or a derivative thereof.

4. The method of claim 3, wherein the perphenazine derivative is SMAP (DT-061) or iHAP.

5. The method as in claim 1, wherein the ARF6 antagonist is: a sphingolipid, a sphingolipid-like compound, NAV2729, SecinH3, or a derivative thereof.

6. The method of any one of claims 3 and 5, wherein the sphingolipid is ceramide, sphingosine, sphinganine, safingol, or other sphingolipid that activates PP2A.

7. The method of any one of claims 3 and 5, wherein the sphingolipid-like compound is based on O-benzyl pyrrolidines having the formula: 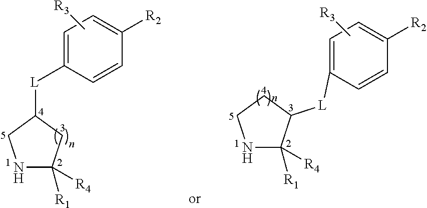
R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, wherein an akyl, alkyne, or alkene is an aliphatic chain up to ten carbons;
R₂ is an aliphatic chain (C₆-C₁₀);
R₃ is a mono-, di-, tri- or quad-aromatic substituent comprising H, halogen, alkyl, alkoxy, azide (N₃), ether, NO₂, or cyanide (CN);
One of R₁ and R₄ is an alcohol (CH₂OH) or H;
L is O—CH₂; and
n is an independently selected integer selected from 1, 2, or 3.

8. The method of any one of claims 3 and 5, wherein the sphingolipid-like compound is based on diastereomeric 3- and 4-C-aryl pyrrolidines having the formula: 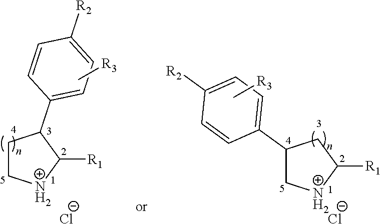
R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, wherein the akyl, the alkyne, or the alkene is an aliphatic chain up to ten carbons;
R₂ is an aliphatic chain (C₆-C₁₄);
R₃ is a mono-, di-, tri- or tetra-aromatic substituent comprising hydrogen, halogen, alkyl, alkoxy, azide (N₃), ether, NO₂, or cyanide (CN); and
n is an independently selected integer selected from 1, 2, or 3.

9. The method of claim 8, wherein the sphingolipid-like compound is compound 893 having the formula:

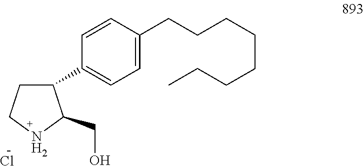 [SMILES: CCCCCCCCc1ccc([C@H]2CCN[C@@H]2CO)cc1 ; Cl]

10. The method of claim 8, wherein the sphingolipid-like compound is compound 1090 having the formula:

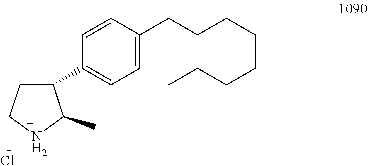 [SMILES: CCCCCCCCc1ccc([C@H]2CCN[C@@H]2C)cc1 ; Cl]

11. The method of any one of claims 3 and 5, wherein the sphingolipid-like compound is based on azacycles with an attached heteroaromatic appendage having the formula: 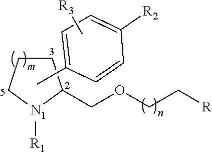
or a pharmaceutically acceptable salt thereof;
R is an optionally substituted heteroaromatic moiety such as an optionally substituted pyridazine, optionally substituted pyridine, optionally substituted pyrimidine, phenoxazine, or optionally substituted phenothiazine.
R₁ is H, alkyl such as C₁₋₆ alkyl or C₁₋₄ alkyl including methyl, ethyl, propyl, isopropyl, n-butyl, s-butyl, isobutyl, t-butyl, etc, Ac, Boc, guanidine moiety.
R₂ is an aliphatic chain comprising 6 to 14 carbons.
R₃ is a 1, 2, 3, or 4 substituents, wherein each substituent, independently, is H, halogen, alkyl, alkoxy, N₃, NO₂, and CN.
n is independently 1, 2, 3, or 4.
m is independently 1 or 2;
the phenyl moiety can be attached at any available position of the azacycle core; and
R is a 1,2-pyridazine having the formula: 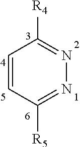
R₄ and R₅ are functional groups independently selected from: alkyl including methyl, optionally substituted aryl (i.e., unsubstituted aryl or substituted aryl) including optionally substituted phenyl, and optionally substituted heteroaryl including optionally substituted pyridine and optionally substituted pyrimidine; and
the pyridazine moiety is connected to the azacycle at the position 4 or 5 of the pyridazine.

12. The method of claim 11, wherein the sphingolipid-like compound is compound 325 having the formula:

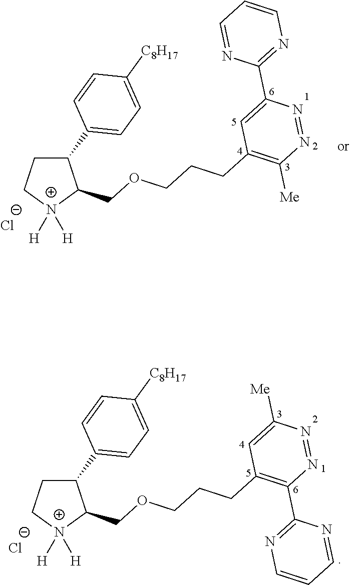

13. The method of any one of claims 3 and 5, wherein the sphingolipid-like compound is based on diastereomeric 2-C-aryl pyrrolidines having the formula: 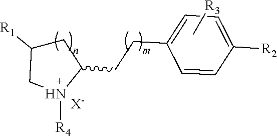
R₁ is a functional group selected from H, an alkyl chain, OH, (CH₂)ₙOH, CHOH-alkyl, CHOH-alkyne, (CH₂)ₙOR′, where R′ is an alkyl, alkene or alkyne.
R₂ is an aliphatic chain (C₆-C₁₄).
R₃ is a mono-, di-, tri- or tetra-aromatic substituent that includes hydrogen, halogen, alkyl, alkoxy, azide (N₃), ether, NO₂, cyanide (CN), or a combination thereof.
R₄ is a functional group selected from H, alkyl including methyl (Me), ester, or acyl.
X⁻ is an anion of the suitable acid.
n is an independently selected integer selected from 1, 2, or 3.
m is an independently selected integer selected from 0, 1 or 2.

14. A method of treating or preventing a viral infection in a subject, comprising:
administering to a subject an antiviral medicament,
wherein the antiviral medicament comprises an agonist of protein phosphatase 2 (PP2A) or an antagonist of ADP Ribosylation Factor 6 (ARF6), and
wherein the subject is infected with an enveloped virus or at risk of infection with an enveloped virus.

15. The method as in claim 14, wherein the antiviral medicament further comprises an antagonist of PIKfyve.

16. The method as in claim 14, wherein the PP2A agonist is: a sphingolipid, a sphingolipid-like compound, perphenazine, a perphenazine derivative, a SET inhibitor, a CIP2a inhibitor, Withaferin A, OSU-2S, FTY720, or a derivative thereof.

17. The method of claim 16, wherein the perphenazine derivative is SMAP (DT-061) or iHAP.

18. The method as in claim 14, wherein the ARF6 antagonist is: a sphingolipid, a sphingolipid-like compound, NAV2729, SecinH3, or a derivative thereof.

19. The method of any one of claims 16 and 18, wherein the sphingolipid is ceramide, sphingosine, sphinganine, safingol, or other sphingolipid that activates PP2A.

20. The method of any one of claims 16 and 18, wherein the sphingolipid-like compound is based on O-benzyl pyrrolidines having the formula: 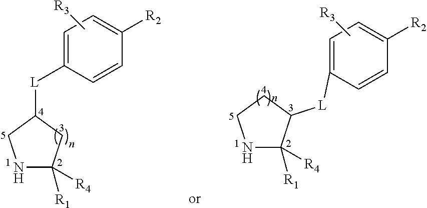
R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, wherein an akyl, alkyne, or alkene is an aliphatic chain up to ten carbons;
R₂ is an aliphatic chain (C₆-C₁₀);
R₃ is a mono-, di-, tri- or quad-aromatic substituent comprising H, halogen, alkyl, alkoxy, azide (N₃), ether, NO₂, or cyanide (CN);
One of R₁ and R₄ is an alcohol (CH₂OH) or H;
L is O—CH₂; and
n is an independently selected integer selected from 1, 2, or 3.

21. The method of any one of claims 16 and 18, wherein the sphingolipid-like compound is based on diastereomeric 3- and 4-C-aryl pyrrolidines having the formula: 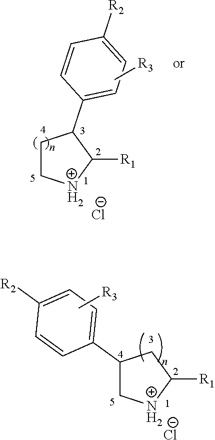
R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, wherein the akyl, the alkyne, or the alkene is an aliphatic chain up to ten carbons;
R₂ is an aliphatic chain (C₆-C₁₄);
R₃ is a mono-, di-, tri- or tetra-aromatic substituent comprising hydrogen, halogen, alkyl, alkoxy, azide (N₃), ether, NO₂, or cyanide (CN); and
n is an independently selected integer selected from 1, 2, or 3.

22. The method of claim 21, wherein the sphingolipid-like compound is compound 893 having the formula:

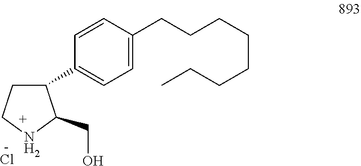 [SMILES: CCCCCCCCc1ccc([C@H]2CCN[C@@H]2CO)cc1 ; Cl]

23. The method of claim 21, wherein the sphingolipid-like compound is compound 1090 having the formula:

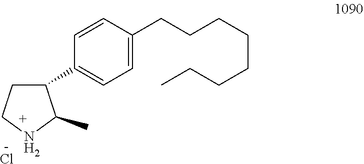 [SMILES: CCCCCCCCc1ccc([C@H]2CCN[C@@H]2C)cc1 ; Cl]

24. The method of any one of claims 16 and 18, wherein the sphingolipid-like compound is based on azacycles with an attached heteroaromatic appendage having the formula: 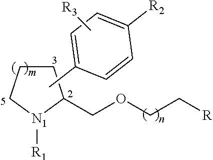
or a pharmaceutically acceptable salt thereof;
R is an optionally substituted heteroaromatic moiety such as an optionally substituted pyridazine, optionally substituted pyridine, optionally substituted pyrimidine, phenoxazine, or optionally substituted phenothiazine.
R₁ is H, alkyl such as C₁₋₆ alkyl or C₁₋₄ alkyl including methyl, ethyl, propyl, isopropyl, n-butyl, s-butyl, isobutyl, t-butyl, etc, Ac, Boc, guanidine moiety.
R₂ is an aliphatic chain comprising 6 to 14 carbons.
R₃ is a 1, 2, 3, or 4 substituents, wherein each substituent, independently, is H, halogen, alkyl, alkoxy, N₃, NO₂, and CN.
n is independently 1, 2, 3, or 4.
m is independently 1 or 2;
the phenyl moiety can be attached at any available position of the azacycle core; and
R is a 1,2-pyridazine having the formula: 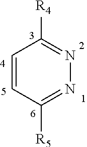
R₄ and R₅ are functional groups independently selected from: alkyl including methyl, optionally substituted aryl (i.e., unsubstituted aryl or substituted aryl) including optionally substituted phenyl, and optionally substituted heteroaryl including optionally substituted pyridine and optionally substituted pyrimidine; and
the pyridazine moiety is connected to the azacycle at the position 4 or 5 of the pyridazine.

25. The method of claim 24, wherein the sphingolipid-like compound is compound 325 having the formula:

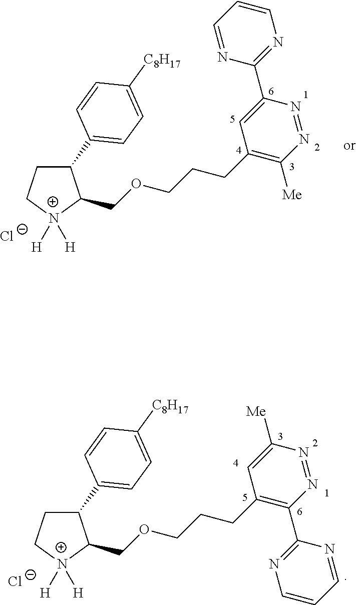

26. The method of any one of claims 16 and 18, wherein the sphingolipid-like compound is based on diastereomeric 2-C-aryl pyrrolidines having the formula: 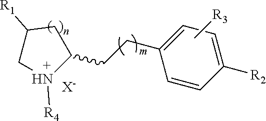
R₁ is a functional group selected from H, an alkyl chain, OH, (CH₂)ₙOH, CHOH-alkyl, CHOH-alkyne, (CH₂)ₙOR′, where R′ is an alkyl, alkene or alkyne.
R₂ is an aliphatic chain (C₆-C₁₄).
R₃ is a mono-, di-, tri- or tetra-aromatic substituent that includes hydrogen, halogen, alkyl, alkoxy, azide (N₃), ether, NO₂, cyanide (CN), or a combination thereof.
R₄ is a functional group selected from H, alkyl including methyl (Me), ester, or acyl.
X⁻ is an anion of the suitable acid.
n is an independently selected integer selected from 1, 2, or 3.
m is an independently selected integer selected from 0, 1 or 2.

27. The use of one or more antiviral compounds in the manufacture of a medicament for the therapeutic treatment of an infection with an enveloped virus,
wherein the one or more antiviral compounds comprises an agonist of protein phosphatase 2 (PP2A) or an antagonist of ADP Ribosylation Factor 6 (ARF6).

28. The use of an antiviral compound as in claim 27, wherein the one or more antiviral compounds comprises an antagonist of PIKfyve.

29. The use of an antiviral compound as in claim 27, wherein the PP2A agonist is: a sphingolipid, a sphingolipid-like compound, perphenazine, a perphenazine derivative, a SET inhibitor, a CIP2a inhibitor, Withaferin A, OSU-2S, FTY720, or a derivative thereof.

30. The use of an antiviral compound as in claim 29, wherein the perphenazine derivative is SMAP (DT-061) or iHAP.

31. The use of an antiviral compound as in claim 27, wherein the ARF6 antagonist is: a sphingolipid, a sphingolipid-like compound, NAV2729, SecinH3, or a derivative thereof.

32. The use of an antiviral compound as in any one of claims 29 and 31, wherein the sphingolipid is ceramide, sphingosine, sphinganine, safingol, or other sphingolipid that activates PP2A.

33. The use of an antiviral compound as in any one of claims 29 and 31, wherein the sphingolipid-like compound is based on O-benzyl pyrrolidines having the formula: 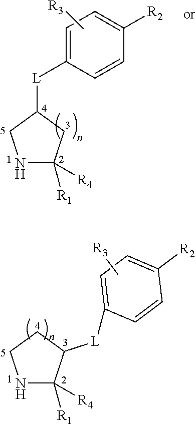
R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, wherein an akyl, alkyne, or alkene is an aliphatic chain up to ten carbons;
R₂ is an aliphatic chain (C₆-C₁₀);
R₃ is a mono-, di-, tri- or quad-aromatic substituent comprising H, halogen, alkyl, alkoxy, azide (N₃), ether, NO₂, or cyanide (CN);
One of R₁ and R₄ is an alcohol (CH₂OH) or H;
L is O—CH₂; and
n is an independently selected integer selected from 1, 2, or 3.

34. The use of an antiviral compound as in any one of claims 29 and 31, wherein the sphingolipid-like compound is based on diastereomeric 3- and 4-C-aryl pyrrolidines having the formula: 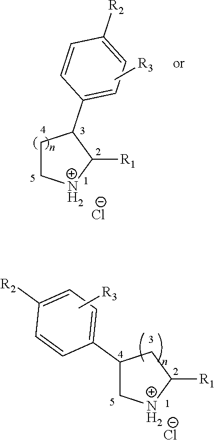
R₁ is an optional functional group selected from an alkyl chain, (CH₂)ₙOH, CHOH-alkyl, CHOH-alkyne, (CH₂)ₙO-alkyl, (CH₂)ₙO-alkene, (CH₂)ₙO-alkyne, wherein the akyl, the alkyne, or the alkene is an aliphatic chain up to ten carbons;
R₂ is an aliphatic chain (C₆-C₁₄);
R₃ is a mono-, di-, tri- or tetra-aromatic substituent comprising hydrogen, halogen, alkyl, alkoxy, azide (N₃), ether, NO₂, or cyanide (CN); and
n is an independently selected integer selected from 1, 2, or 3.

35. The use of an antiviral compound as in claim 34, wherein the sphingolipid-like compound is compound 893 having the formula:

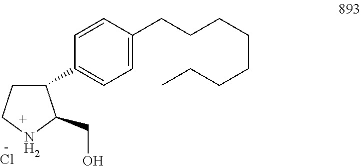 [SMILES: CCCCCCCCc1ccc([C@H]2CCN[C@@H]2CO)cc1 ; Cl]

36. The use of an antiviral compound as in claim 34, wherein the sphingolipid-like compound is compound 1090 having the formula:

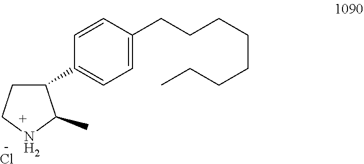 [SMILES: CCCCCCCCc1ccc([C@H]2CCN[C@@H]2C)cc1 ; Cl]

37. The use of an antiviral compound as in any one of claims 29 and 31, wherein the sphingolipid-like compound is based on azacycles with an attached heteroaromatic appendage having the formula: 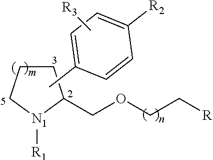
or a pharmaceutically acceptable salt thereof;
R is an optionally substituted heteroaromatic moiety such as an optionally substituted pyridazine, optionally substituted pyridine, optionally substituted pyrimidine, phenoxazine, or optionally substituted phenothiazine.
R₁ is H, alkyl such as C₁₋₆ alkyl or C₁₋₄ alkyl including methyl, ethyl, propyl, isopropyl, n-butyl, s-butyl, isobutyl, t-butyl, etc, Ac, Boc, guanidine moiety.
R₂ is an aliphatic chain comprising 6 to 14 carbons.
R₃ is a 1, 2, 3, or 4 substituents, wherein each substituent, independently, is H, halogen, alkyl, alkoxy, N₃, NO₂, and CN.
n is independently 1, 2, 3, or 4.
m is independently 1 or 2;
the phenyl moiety can be attached at any available position of the azacycle core; and
R is a 1,2-pyridazine having the formula: 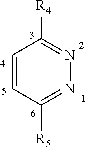
R₄ and R₅ are functional groups independently selected from: alkyl including methyl, optionally substituted aryl (i.e., unsubstituted aryl or substituted aryl) including optionally substituted phenyl, and optionally substituted heteroaryl including optionally substituted pyridine and optionally substituted pyrimidine; and
the pyridazine moiety is connected to the azacycle at the position 4 or 5 of the pyridazine.

38. The use of an antiviral compound as in claim 37, wherein the sphingolipid-like compound is compound 325 having the formula:

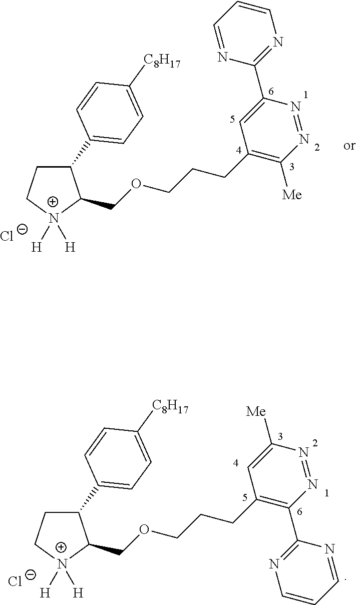

39. The use of an antiviral compound as in any one of claims 29 and 31, wherein the sphingolipid-like compound is based on diastereomeric 2-C-aryl pyrrolidines having the formula: 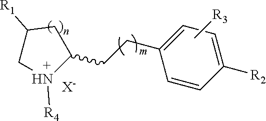
R₁ is a functional group selected from H, an alkyl chain, OH, (CH₂)ₙOH, CHOH-alkyl, CHOH-alkyne, (CH₂)ₙOR′, where R′ is an alkyl, alkene or alkyne. R₂ is an aliphatic chain (C₆-C₁₄).

40. The use of an antiviral compound as in claim 27, wherein the therapeutic treatment is a prophylactic treatment.
